(19) United States
(12) Patent Application Publication
(10) Pub. No.: US 2021/0268127 A1
(43) Pub. Date: Sep. 2, 2021

(54) ENHANCED SURGICAL VISUALIZATION OF VIABLE TISSUE AND PEPTIDES THEREFOR

(71) Applicant: WEST VIRGINIA UNIVERSITY, MORGANTOWN, WV (US)

(72) Inventors: Jonathan W. BOYD, Morgantown, WV (US); Matthew LOOS, Asheville, NC (US)

(21) Appl. No.: 17/204,196

(22) Filed: Mar. 17, 2021

Related U.S. Application Data
(63) Continuation of application No. 15/969,399, filed on May 2, 2018.
(60) Provisional application No. 62/500,350, filed on May 2, 2017.

(51) Int. Cl. A61K 49/00

(52) U.S. Cl. CPC A61K 49/006; A61K 49/0043; A61K 2123/00; A61K 49/0034; A61K 49/0056

(57) ABSTRACT

The various embodiments relate to the identification of non-viable tissue for debridement using a pH low insertion peptide conjugate. pH low insertion peptide conjugates are described having fluorescent dye for introduction to the wound area. The peptides may further be conjugated with a drug for introduction to the tissue.

### CROSS-REFERENCE TO RELATED APPLICATIONS

This application is a continuation of parent U.S. application Ser. No. 15/969,399, filed on May 2, 2018, which claims priority to U.S. Provisional Application No. 62/500,350, filed May 2, 2017. The entire disclosure of each prior application is incorporated by reference herein in its entirety.

### SEQUENCE LISTING

Pursuant to 37 C.F.R. § 1.821(c) or (e), this application contains a sequence listing, which is contained on an ASCII text file entitled “Sequence Listing” (WVU3007-CON_pHLIP_sequences_ST25.txt, created Friday, Mar. 5, 2021, having a size of 1,092 bytes), which is herein incorporated by reference.

### TECHNICAL FIELD

Various embodiments disclosed herein relate generally to methods of visualizing tissue and modified proteins for visualizing tissue.

### BACKGROUND

In the United States, trauma is the leading cause of death in individuals 46 years and younger and has been steadily increasing since 2000. As a point of reference, cancer death rates for all age groups have fallen in the last decade, while trauma death rates have increased dramatically (22.8%) for age groups 25 years and older. The World Health Organization projects that by the year 2020, trauma will overtake infectious disease as the leading cause of death worldwide. Scientific improvements in treating traumatic injuries and improving the natural healing process of tissues are likely to have a significant impact towards decreasing this worrisome trend.

Traumatic wounds heal in a predictive sequence of overlapping phases including inflammatory, proliferative, re-epithelialization and remodeling stages. Success of healing depends on many intrinsic and extrinsic factors that regulate complex biochemical and cellular events, culminating in wound closure via scar tissue formation. Debridement (i.e., removing devitalized tissue, foreign material, senescent cells, phenotypically abnormal/dysfunctional cells, and bacteria sequestrum) has become an essential surgical technique in wound bed preparation because of its demonstrated capabilities to accelerate healing.

Proper surgical debridement is based upon cursory visual inspection of the tissue surrounding a wound to estimate the zone of injury. Defining the zone of injury for traumatic wounds can be difficult, and relies on the surgeon's assessment of coloration of the tissue, consistency and feel of the tissue, contractility of the muscle tissue, and presence of pulsatile flow of the local vasculature. This inexact practice is based on thermal burns, which tend to produce reproducible injuries that are discernable with the naked eye. In thermal burns, the zones of injury are typically well defined: the zone of coagulation (point of maximum damage with irreversible tissue loss), the zone of stasis (little tissue perfusion with potentially salvageable tissue), and the zone of hyperemia (decreased tissue perfusion that is likely to recover). This is in direct contrast to the difficulty of defining a zone of injury in typical trauma cases (e.g. open fracture). Both types of injury require debridement to clear tissue, but the zones of stasis and hyperemia for common traumatic injuries are much more difficult to demarcate.

### Summary of Exemplary Embodiments

Various embodiments recite a method for identifying non-viable tissue for debridement during surgery including administering a pH low insertion peptide conjugated with at least one fluorescent dye to a surgical patient, allowing the conjugated pH low insertion peptide to localize to the tissue and insert into the non-viable tissue, and identifying non-viable tissue marked by the fluorescent dye.

Various embodiments recite the method for identifying non-viable tissue for debridement wherein the tissue marked by the dye is visible to surgeons. In some embodiments, the dye may be a UV fluorescent dye, a NIR fluorescent dye, fluorescein or Indocyanine Green.

Various embodiment recite the method for identifying non-viable tissue wherein the surgical patient is a trauma patient having a wound. In some embodiments the non-viable tissue is at the wound of the trauma patient. The patient tissue marked by the fluorescent dye may be removed from the patient. In some embodiments, the pH low insertion peptide conjugate is injected or applied topically to a surgical patient having a wound.

Various embodiments recite the method for identifying non-viable tissue wherein the pH low insertion peptide conjugate is further conjugated to at least one drug. In some embodiments, the drug is a coagulating agent, anti-bacterial agent, or anti-inflammatory agent.

Various embodiments recite the method for identifying non-viable tissue wherein the pH low insertion peptide has the amino acid sequence according to SEQ ID NO: 1 or 2.

Various embodiments recite a pH low insertion peptide conjugate comprising a pH low insertion peptide and a fluorescent dye, the pH low insertion peptide having the amino acid sequence according to SEQ ID NO: 2.

In some embodiments of the pH low insertion peptide having the amino acid sequence according to SEQ ID NO: 2, the fluorescent dye is a UV fluorescent dye, a NIR fluorescent dye, fluorescein or Indocyanine Green.

Various embodiments recite the pH low insertion peptide having the amino acid sequence according to SEQ ID NO: 2, the pH low insertion peptide is further conjugated to at least one drug. In some embodiments, the drug is a coagulating agent, anti-bacterial agent, or anti-inflammatory agent.

### BRIEF DESCRIPTION OF THE DRAWINGS

In order to better understand various embodiments, reference is made to the accompanying drawings, wherein:

FIG. 1 illustrates the process of binding, folding, and insertion to lipid vesicles by circular dichroism and fluorescence spectroscopy.

FIG. 2 illustrates the factors for successful tissue transfer.

FIG. 3 illustrates Fmoc peptide coupling protocols.

FIG. 4 illustrates method for administering pH low insertion peptide conjugates.

FIGS. 5A, 5B, 5C, 5D, 5E, 5F, and 5G illustrate cytokine concentration vs. time after surgery in a vessel.

FIGS. 6A, 6B, 6C, 6D, 6E, 6F, and 6G illustrate cytokine concentration vs. time after surgery in muscle tissue.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or” refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

Following a traumatic injury on the battlefield, Forward Surgical Teams (FSTs) have many decisions to make in a short time period. The frequency of severe wounds requires FSTs to spend a large amount of their limited resources resolving these issues. Furthermore, extensive wound assessment and treatment options can be limited, while the clock is ticking down on saving tissue and lives. Finding a balance between removal of the entirety of injured tissue, also referred to as debridement, while retaining as much healthy tissue as possible can mean the difference between risking life-threatening infection and saving a limb. Proper wound debridement is critical for the subsequent wound healing processes, and the current method for assessment lacks precision. A solution to this problem is a fluorescently labeled peptide that can elucidate the fine line between healthy and injured tissue.

Wound healing is a complex and fragile biological process, beginning with hemostasis and formation of a platelet plug, then moving to inflammation, when a variety of immune signals are recruited to the wound to prevent infection. During the next phase, proliferation, new tissue grows as multiple skin layers begin to form and cover the wound. Finally, the new skin layers mature, increasing the resilience of the closed wound. Complete wound healing can take up to two years, so it is critical that surgeons protect this delicate process to ensure chronic wounds do not develop. Wound assessment is an integral part of patient success, and clinicians must evaluate the tissue types, check for presence of inflammation and determine the wound edge. Of these criteria, determining wound edge is the most elusive because there are no well-developed methods to quantifiably determine the extent of a wound. Removing the diseased tissue is an essential step in kick-starting wound healing, and successful excision of diseased tissue largely impacts the quality of care. A study has confirmed a strong correlation between performing surgical debridement and improved wound healing rates, showing that in venous leg ulcers and diabetic foot ulcers, debridement procedures improved wound closure progression. More precise debridement would not only drastically improve patient success but would also avoid situations where incomplete debridement introduces new complications for patients. Improper debridement can result in necrotic tissue remaining at the injured site, providing optimal growth conditions for bacteria and creating a nidus for infection. Complete debridement is important for treating abscesses that, when improperly treated, can develop into infections and cause sepsis. A purely visual estimation of wound edge takes more time and expertise while increasing the risk of leaving diseased tissue behind. On the other hand, delayed wound healing can also be a consequence of over-debridement, where too much tissue is removed from the injured site. Finding a medium between these cases is challenging, and FSTs must make these complicated decisions regularly. Currently, there are no standardized guidelines for surgical debridement, as there is a certain amount of artistry involved, requiring years of experience and often consultation with other experts. Attempting to make this evaluation under pressure can increase instances of improper debridement, and this risk necessitates the development of a new tool to assist FSTs in treating severe injuries sustained on the battlefield.

FSTs are mobile units that must move along with the operation, often setting up and providing medical care in dire circumstances. They face a different set of challenges than a typical operating room, encountering patients whose injuries are so severe they cannot be transported to a care facility without immediate surgical intervention. FSTs do not have time to waste and often mistakes can result in severe tissue loss or death. It is necessary to respond to the needs of FSTs by improving surgical technology to alleviate time pressures. A shift to a mobile, low cost and user friendly diagnostic tool will allow more confident decisions to be made by FSTs regarding debridement, leading to an increase in tissue preservation. Furthermore, the technology is very intuitive, as it will be available by administering an intravenous injection or a topical solution. Health care providers would not need excessive training to become familiar with its use, and it will easily be integrated into existing technologies, therefore not imposing high costs for transition.

As warfare technology has progressed to more sophisticated and deadly tactics, prevalence and severity of traumatic injury have increased. During Operation Iraqi Freedom and Operation Enduring Freedom, 78% of wounds sustained in combat were a consequence of explosions, and extremities are the most commonly affected body region. Due to the severity and frequency of these types of injuries, FSTs must think, move and act more quickly to keep pace with patients' needs because mistake could result in amputation and major tissue loss. A study investigating the frequency and nature of injuries treated by FSTs in Afghanistan during the initial phase of Operation Enduring Freedom found that debridement was the most common procedure performed. This trend can be expected to continue as the use of explosives in combat continues to rise, meaning that wound assessment will continue to be a time-consuming and complicated treatment step if new methods are not implemented. To accommodate the need for more precise treatment, new surgical techniques can be developed to incorporate fluorescent probes that assist surgeons' assessments of wounds. These advances would not only alleviate time pressure but would improve the quality of care given to patients by establishing a clear, quantifiable method for wound edge determination. Fluorescent surgical techniques can provide the foundation for these advances.

To enhance the ability of surgeons and FSTs to differentiate viable from non-viable tissue, an exemplary embodiment is directed to a peptide-fluorophore conjugate that will preferentially bind to injured tissue based upon spatial pH gradients. Relatively low cellular pH levels within the superficial zone of injury (above the dermal layer) have been known since the 1970s, but the relationship between the pH of deeper tissues (e.g. musculoskeletal tissues) and wound healing have not been well characterized. For deep muscle tissue, researchers have demonstrated that a spatially-dependent decrease in pH (from ˜7.2 to ˜6.6) occurs immediately following surgical incision and lasts for up to 4 days. Another researcher was able to demonstrate spatial pH changes of traumatic injuries in si and related it to rates of infection, but not tissue viability.

An exemplary embodiment further includes a viable peptide-conjugated fluorescent dye that can be used by orthopedic surgeons as a topical solution; one that is capable of providing easy spatial discrimination of injured tissues and a dye that can be injected upstream of injured tissues (upon presentation to the trauma center). The dye will flow through the microvasculature and insert preferentially into low pH tissue, allowing all surgeons facile visual determination of the complete zone of injury. An exemplary embodiment may further include peptide conjugates to additional diagnostic markers, such as inflammatory response cytokines, hormones, and growth factors.

Several classes of cell-penetrating peptides have been developed for use as antimicrobial peptides and in drug delivery; however, the majority function by a two-step process of cell surface aggregation and pore formation to facilitate cell death or drug delivery. The pH-Low Insertion Peptide (pHLIP) is unique in the sense that it acts as a single transmembrane peptide with acid-sensing capability.

At the molecular level, pH low insertion peptide reversibly transitions between three states: in solution it exists as an unfolded peptide (state I); upon exposure to the bloodstream it binds indiscriminately to cell surfaces (state II); and if bound to a cell surface with an acidic exterior microenvironment, it undergoes folding into a helical conformation and unidirectional insertion into the cell membrane (state III). An increase in the rate of glycolysis within the cells of injured tissues causes a lowered local pH environment, which allows the peptide to insert into cell membranes. This mechanism makes pH low insertion peptide a potentially viable vehicle for targeted diagnostic imaging of biomedical issues associated with acidosis.

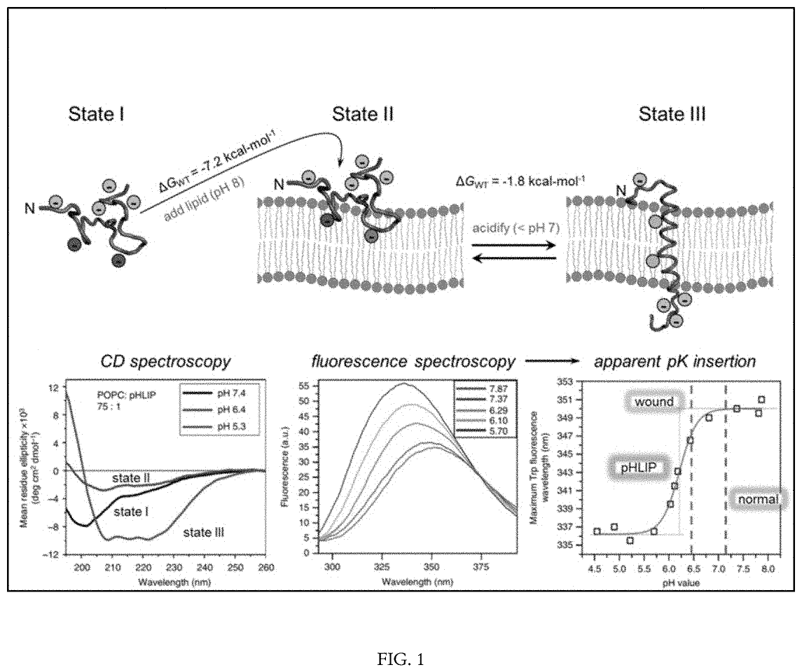

The acid-sensing capability of pH low insertion peptide is tied to two interior acidic amino acid residues (D14 and D25) that undergo protonation upon acidification, triggering folding and insertion mechanisms. The process of binding, folding, and insertion to lipid vesicles by circular dichroism and fluorescence spectroscopy is illustrated in FIG. 1.

ph low insertion peptide exists in three conformational states: the first is the free-floating monomeric peptide in solution. If lipids are introduced into the environment at normal physiological pH, the peptide will loosely associate with lipid membranes and adopt another conformation. When pH dips below physiological levels, pH low insertion peptide assumes a coiled conformation and inserts across the lipid membrane; this phenomenon presents an opportunity for pH low insertion peptide applications to cancer, injuries and more. A pH drop causes protonation of aspartic acid and glutamic acid residues, increasing the peptide's affinity for lipid bilayers and promoting insertion. Upon insertion, the C-terminus penetrates the cell while the N-terminus remains outside. Attaching fluorescent tags to the N-terminus of the peptide, may allow real-time monitoring of the injury. The affected area can then be visualized to elucidate the zone of injury. Development of fluorescent conjugated pHLIP-like peptides would provide FSTs a novel tool to help them make quick decisions in the field regarding wound severity and allow quick access to realistic treatment options. In an exemplary embodiment, a product could be seen with the naked eye and be used practically anywhere. An accelerated assessment tool comprised of a conjugated peptide of an exemplary embodiment could save lives and increase efficiency of resources in the field.

Developments in fluorescence-assisted surgery have led to advances in the operating room, equipping surgeons with more precise diagnostic tools. Using fluorescent tags improves the surgeon's evaluation by targeting tissue that needs to be removed and creating a clear visual boundary between diseased and healthy tissue. Combining this tool with imaging instrumentation allows real-time assistance for surgeons.

Two versions of pH low insertion peptide which may be used in the described process include: 1) wild-type (wt-pHLIP), the 37-residue peptide that was originally discovered to have acid-sensing capabilities; and 2) pHLIP-1, a 32-residue variant that shows significantly faster kinetics of insertion into vesicles. Both peptides have similar pK's of insertion (i.e., the pH (˜6.2) at which 50% of pH low insertion peptide is inserted into a cell membrane); however, wt-pHLIP inserts into membrane-like phospholipid vesicles in 32 s whereas pHLIP-1 inserts in 80 ms.

wt-pHLIP may be characterized by the following protein sequence designed SEQ ID NO. 1: ACEQNPIYWARYADWLFITPLLLLDLALLVDADEGCT

pHLIP-1 may be characterized by the following protein sequence designed SEQ ID NO. 2: ACEDQNDPYWARYADWLFITPLLLLDLALLVGT

wt-pHLIP is capable of carrying imaging agents attached to the N-terminus of the peptide and localizing to acidic tissues such as those present in injured tissues. Several classes of fluorescent molecules have been successfully used in conjunction with wt-pHLIP to image cells (¹⁸F-based molecules incorporated through click chemistry), tumor tissues (rhodamine and BODIPY), and mice (Alexa fluorophores). Localization of inserted pH low insertion peptide to cell samples has been shown to occur within 20 minutes; which is appropriate for topical application to wound debridement. In an exemplary embodiment, Fluorescein-5-maleimide may be an imaging agent conjugated to pHLIP-1. It is one of two FDA-approved imaging agents and is characterized by lower cost but with the drawback of reduced sensitivity against biological background. However, it may provide enough fluorescence to facilitate imaging with available CCD cameras that are already used in surgical settings.

Two of the top five most common surgical procedures in the United States, joint replacement and broken bone repair, typically involve definition of the zone of injury and debridement of tissue. More than 1 million Americans have joint replacement surgery annually, and all require debridement and discrimination of viable and non-viable tissues. The cost of joint replacement ranges from $16,000-$60,000 in the U.S. In addition, in 2015 there were nearly 700,000 surgical procedures performed for repair of broken bones (average cost: $8,000); the amount of debridement for these procedures varies, but discrimination of tissue viability and definition of the zone of injury is paramount to successful treatment. The pHLIP-1-dye for both formulations, topical and injectable, is intended to be identical.

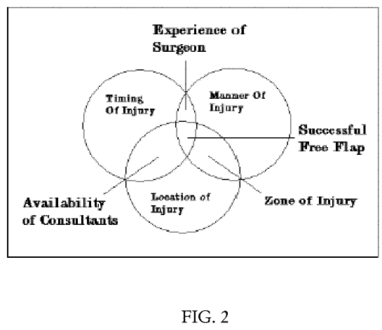

Further, the birth of the microvascular revolution during the 1960's and 1970's led to the new ability to cover wounds across the body with a technique known as a free flap surgical procedure. This procedure typically involves taking tissue from one part of the body and attaching it to another part of the body (e.g. replacing damaged body vessels to enable blood flow to the extremities). However, these flaps were faced with an exceedingly high failure rate of up to 20%. In an attempt to describe an apparent source for this high failure rate, there was recognition during the 1980's of a “zone of injury” that seemed to surround a gross tissue defect. A distillation of the body of literature currently existing on the success of free tissue transfer lends itself to the diagram in FIG. 2. The illustration indicates a lack of diagnostic tests regarding the overall damage, nor are there any means to determine or predict viable vs. non-viable tissue in the zone of injury. The overall lack of diagnostics to define the zone of injury leaves the surgeon to rely solely on experience, and the primary method for determining the usability of recipient vessels is based on the visual inspection of the vessels under the microscope as discussed above in regard to field operations. The surgeons are looking for the presence of pulsatile flow within the recipient vessel, but it is known that the vessel epithelial cells within the zone are still subject to death despite the presence of pulsatile flow. There are no studies looking at a reproducible means for determining or predicting reliable tissue or cell viability within or beyond the visual zone of injury. As experience and specialization grow in the use of free flaps for treatment of traumatized extremities, the zone of injury must be thought of as an integral part of the success of free tissue transfer and a greater understanding will likely help to improve the outcomes of these patients.

To increase the success rate of free flap surgeries, appropriate identification of the zone of injury and its relationship to tissue viability must be determined. Organizationally, tissue death is preceded by cell death and traditionally cell death has been categorized as either necrosis or apoptosis. Necrosis is often associated with acute injury and in general rapidly affects tracts of contiguous cells in the damaged tissue. In contrast, apoptosis has usually been associated with chronic or delayed cell injury and is the morphological result of a relatively slow process often occurring over several hours or days. Unlike necrosis, cellular metabolism and membrane integrity are maintained until a very late stage of the process leading to apoptotic cell death, which makes visual inspection of tissues an unreliable means of predicting long-term viability. Apoptotic-related mitochondrial mechanisms of cell mortality are most commonly cited as explanations for secondary injury following traumatic and burn injuries.

Apoptotic initiation is expected to be related to either the extrinsic (extracellular) or intrinsic (intracellular) pathways of cell death. The extrinsic pathway initiates apoptosis via transmembrane receptor-mediated interactions and involves death receptors that are member of the tumor necrosis factor (TNF) receptor gene superfamily (e.g. FasL/FasR, TNF-alpha/TNFR1, etc.). Alternatively, the intrinsic signaling pathways that initiate apoptosis involve a diverse array of stimuli (e.g. DNA damage, inflammation, etc.) that produce intracellular signals that act directly on targets within the cell. It is well established that there is a long pro-inflammatory cytokine response following severe injury that involves increased serum levels of Interleukin-1 (IL-1), IL-2, tumor necrosis factor-alpha (TNF), IL-6, IL-12, and Interferon-gamma (IFN), which can either initiate apoptosis directly (e.g. TNF) or can affect inflammation (e.g. IL-6). However, much less is known with regards to local inflammation following injury and its role in apoptosis. It has been demonstrated that blunt proximal trauma (e.g. closed femoral fracture) greatly induces activation IL-6 and IL-8 in subcutaneous adipose tissue near the injury as compared to remote tissue. Further, a study of local pain revealed that the increased combinatorial presence of four cytokines (IFN, IL-6, monocyte chemotactic protein-1 (MCP-1, and macrophage inflammatory protein-1 beta (MIP-1b)) taken from local fluid samples were 100% predictive of meniscal tears. These studies suggest that there may be both temporal and spatial biomarkers that could be used to predict tissue viability.

ph low insertion peptide conjugate use is a promising new technology for surgical use in real-time wound assessment. However, more research may indicate how pH low insertion peptide conjugates respond to different cellular environments. To simulate real biological systems, in vitro models using L6 Rat myoblasts may be employed. These skeletal muscle cells are optimal for replicating the conditions of a wound. After the cells grow and fully differentiate into fibroblasts—which have the characteristics of smooth muscle tissue-they can be exposed to acidic or basic environments. The acidic conditions are essentially a simulated injury representative of a wound while basic environments can serve as a control to ensure that pH low insertion peptide will not insert into the membrane at higher pH values. The cells may be used to study the interactions of pH low insertion peptide as a function of concentration across a pH range to establish a relationship between signal and pH. An understanding of how pH low insertion peptide will react in different environments may also apply to human systems. Additionally, the model may explain the aggregation patterns of pH low insertion peptide. Current research suggests that pH low insertion peptide is not in danger of aggregating below 50 μM, and the propensity of aggregation can be tuned by altering the sequence of pH low insertion peptide. Aggregation studies may determine what concentration of pH low insertion peptide should be used to find a balance between amount of signal and clearance time.

An exemplary embodiment may include pHLIP-1, which inserts more quickly than other developed variants. Altering the insertion speed through sequence modifications may provide proteins for different clinical applications, for example topical and/or injectable applications. pH low insertion peptide technology allows for this type of flexibility by changing the tag on the peptide. FSTs could employ UV fluorescent or NIR fluorescent dyes, and there are advantages to each. A UV fluorescent dye like fluorescein would provide a cheap, easy-to-use product that could be transported and used almost anywhere. NIR fluorescent dyes, like Indocyanine Green (ICG) can be used for more sensitive detection, as they reduce the amount of white light interference. Their use would require implementing already existing technologies in hospitals to image the NIR signal. With either embodiment, an injectable solution of pH low insertion peptide may be visualized rapidly.

pH low insertion peptide products are not limited to wound identification and can potentially develop into applications for wound treatment. The drug delivery capabilities of pH low insertion peptide have been investigated, and the peptide was shown to possess the ability to deliver cell-impermeable cargo across the plasma membrane. pH low insertion peptide has dual delivery potential, as it can tether small molecules to the plasma membrane surface, which is the principle behind using the attached fluorescent probes for injury site identification. Drug delivery feasibility is often guided by Lipinski's rule of five which suggests drugs should be small and hydrophobic to reach intracellular targets. Many biological inhibitors cannot pass through a membrane unassisted, but pH low insertion peptide can translocate these agents with ease. pH low insertion peptide has successfully been shown to deliver nanogold particles to tumor sites via intravenous injection, allowing for “controlled” toxicity to tumors, and the peptide is used as a vehicle for nanomedical treatment of cancers. More traditionally, the peptide can inject and release small molecules inside the cell. When small molecules are attached to the C-terminus of the peptide, they are transported inside the cell. The reducing environment of the cytosol results in disulfide bond cleavage, triggering a release of the cargo. pH low insertion peptide conjugates can be used for drug delivery to tumor sites, and pH low insertion peptide shows no toxicity to cells at concentrations below 50 NM. Several studies have been conducted to confirm that pH low insertion peptide will release its cargo inside the cytosol. A study of the translocation of phalloidin, a toxic agent that inhibits tumor growth, confirmed the ability of the peptide to effectively deliver cargo. When the toxin was attached to the C-terminus and exposed to cells in acidic environment, it prohibited tumor proliferation. These findings confirmed pH low insertion peptide viability for drug delivery.

Considering the drug delivery capabilities of pH low insertion peptide, there may be several applications for wound healing. pH low insertion peptide has already been shown to transport cell-impermeable molecules across plasma membranes to treat tumors, so it is not a far reach to apply this technology for wounds. For drug delivery applications, cell-impermeable cargo can be attached to the peptide's C-terminus, as stated previously. The cargo is attached via a disulfide bond that can subsequently be cleaved upon insertion, releasing the cargo into the cytoplasm where it can act therapeutically.

One of the most challenging combat injuries that FSTs face is deep bleeding wounds, such as large chest cavities. These types of wounds cannot be treated with a tourniquet and result in major blood loss. Currently, there is no way to deal with this deep bleeding other than to physically restrict blood flow. Cauterizing and pinching off vessels are the only options but are usually ineffective, as it is extremely difficult to deal with the extent of the cavity. pH low insertion peptides may be used to introduce coagulating agents to essentially pinch off arteries and create a sort of molecular tourniquet. For example, during the inflammatory phase of wound healing, peroxisome proliferator-activated receptor β (PPARβ) acts as an anti-apoptotic factor, so survival of cells in the affected area depends on its activation. Peptide delivery of activating factors early on can potentially provide a way to induce wound healing processes at the cellular level earlier than the body would normally be able to accomplish. These techniques could be used to affect homeostasis. The dynamics of pH low insertion peptide support that it can locate injuries and insert in a few seconds. So, this type of treatment could act quickly to prevent major blood loss and assist the body's efforts to begin wound healing. Because all pH low insertion peptides insert at low pH, it would be possible to deliver a cocktail of different agents to the site, including anti-bacterial and anti-inflammatory agents to reduce the severity of the injury, further assisting the homeostatic mechanism.

One of the challenges faced by FSTs is the pressure to make quick decisions about wound treatment in a short amount of time, but current methods involve time-consuming treatment by highly trained clinicians. Fluorescent pH low insertion peptide products would free up resources but cutting down on time spent for debridement evaluation. The key to effective treatment is adaptability to multiple situations. However, it is costly and time-consuming to develop mobile technologies that allow FSTs to keep pace with combat units. A pH low insertion peptide based conjugate may provide an affordable, easy-to-transport tool that is flexible enough to assist in a wide range of medical cases. Different peptide variants can be designed with a variety of fluorophores or cargo molecules to meet the specific needs of all types of circumstances. By developing pH low insertion peptide conjugated products, FSTs will gain access to new treatments for combat wounds, spanning from more precisely identifying the wound edge for debridement to delivery of wound-healing factors that kick start the healing process. This improved accuracy and broadened scope will lead to tissue preservation, decreased rates of infection and enhanced quality of care.

### Example 1

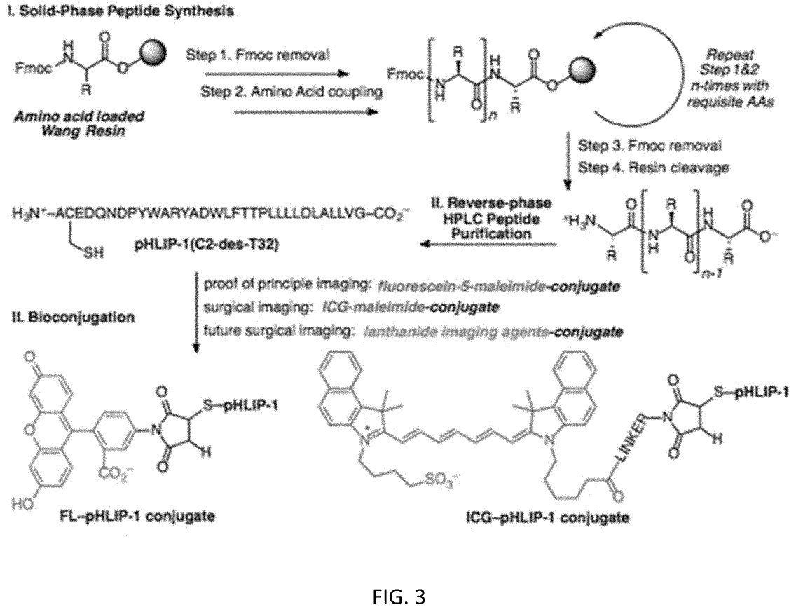

ph low insertion peptides are prepared using standard Fmoc peptide coupling protocols (Step 1-4, FIG. 3) with pre-loaded Wang resin applied to the synthesis of a pHLIP-1 derivative in which a cysteine residue (2C) is inserted and the C-terminal threonine residue is deleted. Subsequent crude peptide purification to >90% purity is achieved using an optimized semi-preparative HPLC method with a Phenomenex Jupiter Proteo 90 Å column that is engineered specifically for separations of proteins and peptides<10,000 Da, allowing reduced solvent consumption. Conservatively, each synthesis yields 160-400 mg (0.04-0.1 mmol) of pHLIP-1 under an initial unoptimized coupling protocol. Further synthetic elaboration of pHLIP-1 will be achieved through site-selective reaction of the cysteine residue with fluorescein-5-maleimide, a fluorescent imaging agent. This peptide synthesis research strategy is well suited for future targeted optimization of both the peptide sequence, to achieve improved solubility and cell-membrane insertion resonance time, and bioconjugation substrate, to obtain better surgical imaging (e.g. FDA-approved near-infrared fluorophore ICG). Lanthanide-based imaging agents that show significant promise for surgical imaging, owing to large excitation/emission wavelength separation, sharp emission bands, and long luminescence life-times (ms) may be integrated with the peptide.

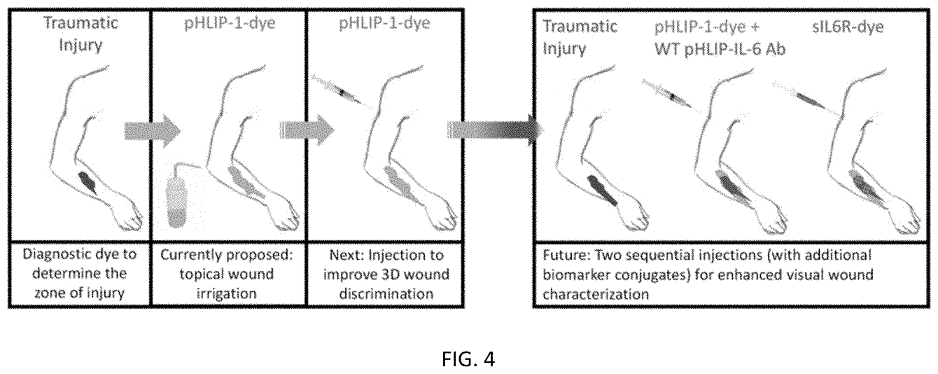

Visualization and forecasting viable tissue may be enhanced based on colocalization with known biomarkers. Enhancing visualization may be accomplished by altering the delivery of the pHLIP-1 dye. An exemplary embodiment may thereby include a topical solution with no-low side effects, which may yield favorable responses from patients and surgeons. An exemplary embodiment may include the conjugate in an injectable solution. An injectable solution may be desirable because it may allow facile discrimination of the zone of injury in three-dimensional space, drastically decreasing the need to reapply the dye as tissue is removed. In order to forecast tissue viability in a traumatic injury, biomarkers that are early predictors of cellular death should be measured and visualized within the zone of injury. One such biomarker is IL-6, which can be readily measured with selective antibodies. An antibody (Ab)-based enzymatic system may be utilized that will occur in two steps: 1) injection with pHLIP-1-dye (that will outline the zone of injury) and a wild type (WT) pH low insertion peptide that is conjugated to an antibody specific for IL-6 (that will insert into lower pH tissues that have a lower probability of survival); 2) injection with soluble IL-6 receptor that is conjugated to an alternatively colored dye (that will bind to IL-6 in the blood stream and accumulate around tissue that is forecasted to be nonviable within 24 hours). A general schematic of a potential process is outlined in FIG. 4.

### Example 2

To better understand the local response to blunt trauma, several cytokines from tissue samples taken from rats following a Gustillo III-b injury (open femoral fracture) were measured. GM-CSF, IL-1a, IL-1b, IL-2, IL-6, MIP-1a, and TNF-alpha, concentrations were measured from blood vessels and muscle samples directly at the site of fracture, 1 cm away from fracture, and from the opposite, non-injured leg; samples were obtained at 0, 6, 24, and 168 hours post-fracture. Cytokine targets cast a wide net on overall activity and represent mediators of apoptosis, as well as pro- and anti-inflammatory agents as shown in the table above. Overall, tissue-dependent variations in cytokine concentrations appear to be both temporally and spatially regulated. These findings represent new biomarkers of the zone of injury and may provide the framework for developing future diagnostics that could be used by surgeons to quickly distinguish viable/non-viable tissue.

Adult male rats were housed individually with a 12:12 light/dark cycle and ad libitum access to food and water. Twelve animals were used for the study (3 animals per time point, 4 time points). After adequate anesthesia all animals were subjected to a standardized femur fracture on one leg using a custom designed tool in which a weight is dropped in a consistent fashion onto the mid-shaft of the rat's thigh. Buprenorphine SR was pre-operatively administered subcutaneously as an analgesic providing 72 hour pain relief. Rats were anesthetized intraperitoneally with Ketamine (80-90 mg/kg) and Xylazine (10-15 mg/kg). Using sterile technique and instruments, an incision was made to visualize the fracture. A hole was drilled into the proximal femur to allow a 0.045 inch K-wire to be inserted down the intramedullary canal to fix the fracture. The wound was closed starting with the fascia and then using a stainless steel suture on the skin. Rats were subcutaneously administered Yohimbine (2 mg/kg) post-operatively to reverse the xylazine and were closely observed during recovery for signs of distress. Rats were divided into groups of 3 to be sacrificed at 4 times points following the surgery. At the appropriate time rats were anesthetized intraperitoneally with Ketamine (80-90 mg/kg) and Xylazine (10-15 mg/kg). One cc of Euthasol was then administered via intracardiac puncture as approved by the American Veterinary Medical Association.

Rats were sacrificed immediately following leg fracture, 6 hours after leg fracture, 24 hours after leg fracture, and 7 days (168 hours) after leg fracture. Following sacrifice, blood vessels and muscle tissue was harvested from the site of the fracture, 1.0±0.02 cm away from the site of fracture, and from the leg opposite to the fractured leg. Once removed, samples were processed. Following harvest samples were immediately rinsed with ice cold phosphate buffered saline, snap frozen and stored at −80° C. Samples were ground cryogenically and then lyophilized for 48 hours at 0.080 mBar and −90° C. For analyses, 2-3 mg of ground and lyophilized tissue samples were weighed and thawed for 10 min at 4° C. in 800 μl (muscle samples) or 650 μl (vessel samples) of cell lysis buffer containing 20 mM phenyhlmethylsulfonyhl fluoride. Thawed samples were then vortexed for 1-3 sec and homogenized with 3 rapid pulses using a model 100 ultrasonic dismembrator. Samples were vortexed for 1-3 sec and centrifuged at 5,000×g for 5 min at 4° C. The supernatant was then collected and total protein concentration was determined for each sample using the RC DC protein assay according to the manufacturer's instructions. Absorbance values were determined using an Infinite M1000 plate reader. All samples were diluted to a final total protein concentration of 900 μg/ml with sample diluent.

Sample homogenates were assayed for cytokines using the BioPlex Pro multiplexed GM-CSF, IL-1a, IL-1b, IL-2, IL-6, TNF-α and MIP-1a magnetic bead-based immunoassay reagent kit along with the BioPlex 200 suspension array system and Pro II Wash Station according to the manufacturer's instructions. Data were analyzed using Prism 5. A five-parameter logistic regression model was used to create a standard curve for each protein and determine sample cytokine concentrations. Cytokine concentrations were expressed as nanogram of cytokine per gram of total protein in sample. Analysis of variance with Bonferroni's post test was used to determine significant differences between each sample distance of each of the 4 time points. Data were expressed as the mean±standard error of the mean.

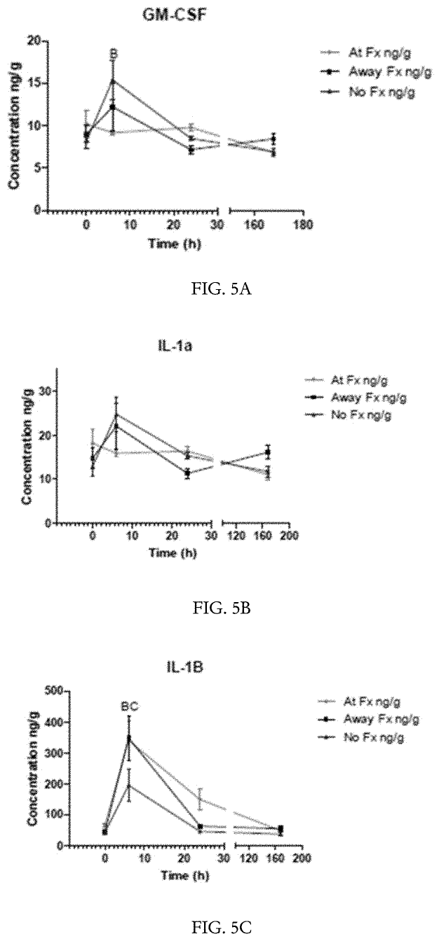

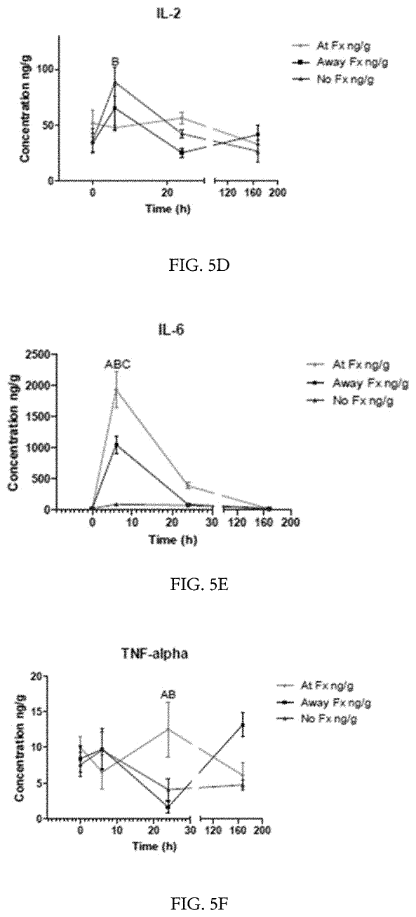

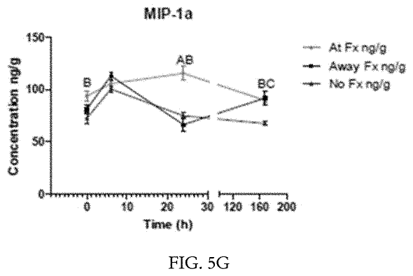

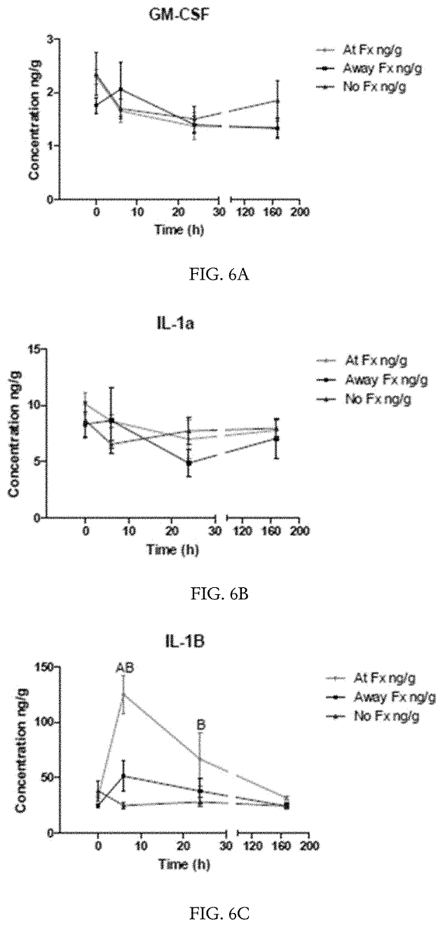

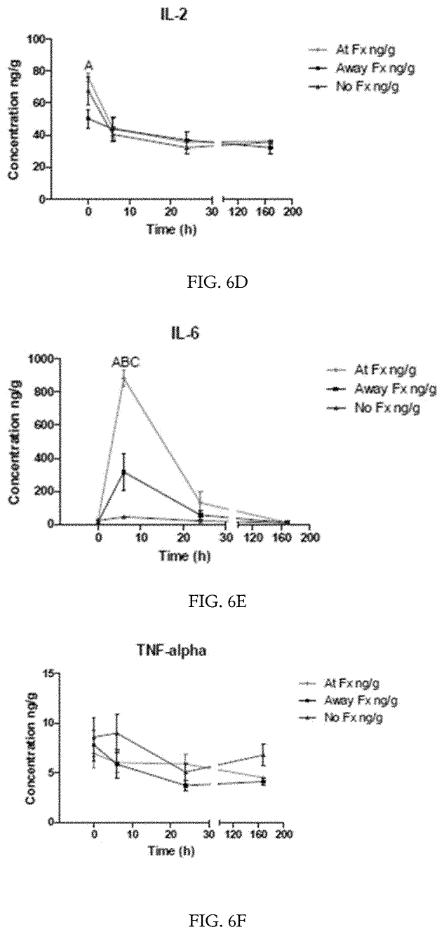

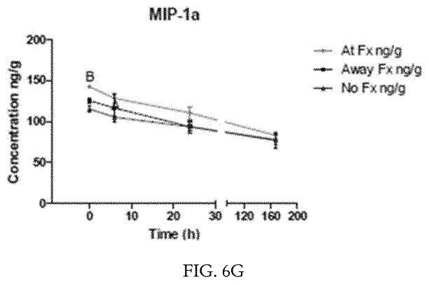

FIGS. 5A-5G show changes in protein concentration at each time point for each of the 3 distances from the fracture site for the vessel samples while FIGS. 6A-6G shows the same for the muscle samples. At Fx represents samples taken directly from the fracture site. Away Fx represent samples taken 1.0 com from the facture site. No Fx represent samples taken from the leg opposite to the facture leg. Concentrations are expressed as nanogram of cytokine per gram of total protein. Points marked with A, B, or C represent statistically significant differences in concentrations between different sample distances. IL-6 levels were significantly different (P<0.001) at 6 hours post surgery at all 3 sites in both vessel and blood samples showing increased levels closest to the fracture site. In vessel samples, IL-1b levels at 6 hours were significantly different (P<0.05) when comparing the site of the fracture to no fracture and also between samples away from the fracture and with no fracture. In muscle samples, IL-1b levels at 6 hours post surgery were significantly different (P<0.001) at the fracture vs. away from the fracture as well as at the fracture vs. no fracture. Muscle IL-1b levels were also significantly different (P<0.05) at 24 hours when comparing at fracture to no fracture samples. IL-2 levels in vessel samples were significantly different (P<0.05) at 6 hours when comparing at fracture to no fracture and when comparing away from fracture to no fracture. In muscle samples IL-2 levels were significantly different (P<0.01) 0 hours after surgery when comparing samples at the fracture to those away from the fracture. MIP-1a levels in vessels were significantly different at 0 hours when comparing at fracture to away from fracture (P<0.001), at 24 hours when comparing at fracture to away from fracture (P<0.001), 24 hours at fracture vs. no fracture (P<0.001), 168 hours at fracture vs. no fracture (P<0.05), and 168 hours away from fracture vs. no fracture (P<0.01). In muscle samples, the only significant difference in IL-2 levels was found at 0 hours at fracture vs. no fracture (P<0.05). TNF-alpha levels in blood vessels were significantly different at 24 hours when comparing at fracture to away from fracture (P<0.05) and at fracture to no fracture (P<0.01). There were no significant differences in TNF-alpha levels for all muscle samples. Vessels samples showed a significant difference in GM-CSF levels at 6 hours when comparing samples at the fracture to those with no fracture (P<0.01). There were no significant differences in muscle GM-CSF levels for any of the treatment groups. There were also no significant differences in IL-1a levels for any of the treatment groups in either vessel of muscle samples.

The lack of data that links the temporal and spatial domains of inflammatory response to localized traumatic injury has limited understanding of the zone of injury. This example defined the zone of injury using a reproducible injury model and monitored inflammatory cytokine concentrations found in blood vessels and surrounding muscle tissues at sites both near and distal to the injury and at several time points post-fracture. Both spatial and temporal-dependent concentrations of IL-1b, IL-6, and MIP-1a were found in both sample tissues. Vessels, which can both deliver and produce these cytokines, are key to successful free flap surgery. GM-CSF, IL-1b, IL-2, IL-6, TNF-alpha, and MIP-1a were all found to be significantly different from the uninjured leg. The vessel responses that were found to be significantly different from the non-injured leg spanned each time point (0, 6, 24, and 168 hours post-fracture), which will allow the zone of injury to be expanded from a spatial concept into the temporal domain. The muscle samples provided further confirmation that the high concentrations of IL-1b and IL-6 in the vessel were perfusing into local tissue. The muscle samples also point to the potential that MIP-1a concentrations (which were higher in the muscle than in the vessel) are actually being secreted from the muscle, rather than from the vessel at 0 hours. Overall, key biomarkers of inflammation are spatially and temporally regulated in response to traumatic injury.

The biomarkers of inflammation may be related to tissue viability and apoptosis. The amount of cleaved PARP and CASPASE 3, intracellular proteins that are intricately involved in apoptosis, within samples may be determined. Without being bound to theory, it is postulated that cytokine concentrations will be closely associated with the relative amounts of apoptotic markers. Since IL-6 has been shown to be both pro- and anti-inflammatory, its relationship with cleaved PARP and CASPASE 3 may be both concentration and time-dependent. Further IL-1b, IL-6 and MIP-1a may be linked with downstream intracellular targets that can be predictive of cell viability.

1. A method for identifying non-viable tissue for debridement during surgery comprising:
administering a pH low insertion peptide conjugated with at least one fluorescent dye to a surgical patient,
allowing the conjugated pH low insertion peptide to localize to the tissue and insert into the non-viable tissue, and
identifying non-viable tissue marked by the fluorescent dye.

2. The method of claim 1, wherein the surgical patient is a trauma patient having a wound.

3. The method of claim 2, wherein the non-viable tissue is at the wound of the trauma patient.

4. The method of claim 1, wherein the patient tissue marked by the fluorescent dye is removed from the patient.

5. The method of claim 1, wherein the fluorescent dye is selected from the group consisting of a UV fluorescent dye and a NIR fluorescent dye.

6. The method of claim 1, wherein the fluorescent dye is selected from the group consisting of fluorescein and Indocyanine Green.

7. The method of claim 1, wherein the pH low insertion peptide conjugate is injected into a surgical patient having a wound.

8. The method of claim 1, wherein the pH low insertion peptide conjugate is topically applied to a surgical patient having a wound.

9. The method of claim 1, wherein the pH low insertion peptide conjugate is further conjugated to at least one drug.

10. The method of claim 9, wherein the at least one drug is selected from the group consisting of coagulating agents, anti-bacterial agents, and anti-inflammatory agents.

11. The method of claim 1, the pH low insertion peptide having the amino acid sequence according to SEQ ID NO: 1 or 2.

12. The method of claim 11, the pH low insertion peptide having the amino acid sequence according to SEQ ID NO: 2.

13. A pH low insertion peptide conjugate comprising a pH-low insertion peptide (pHLIP) and a fluorescent dye, the pH low insertion peptide having the amino acid sequence according to SEQ ID NO: 2.

14. The pH low insertion peptide conjugate of claim 13, wherein the fluorescent dye is selected from a UV fluorescent dye and a NIR fluorescent dye.

15. The pH low insertion peptide conjugate of claim 13, wherein the fluorescent dye is selected from fluorescein and Indocyanine Green.

16. The pH low insertion peptide conjugate of claim 13, wherein the pHLIP conjugate is further conjugated to at least one drug.

17. The pH low insertion peptide conjugate of claim 16, wherein said at least one drug is selected from the group consisting of coagulating agents, anti-bacterial agents, and anti-inflammatory agents.

18. A method for debriding non-viable tissue during surgery, comprising:
identifying non-viable tissue for debridement during surgery by the process of claim 1, and
debriding the non-viable tissue.

19. A method for debriding non-viable tissue during surgery, comprising:
administering a pH low insertion peptide conjugated with a fluorescent dye to a surgical patient, wherein the pH low insertion peptide has the amino acid sequence according to SEQ ID NO: 1 or SEQ ID NO: 2;
allowing the pH low insertion peptide conjugated with the fluorescent dye to localize to the tissue and insert into the non-viable tissue;
identifying non-viable tissue marked by the fluorescent dye; and
debriding the non-viable tissue.


SEQUENCE LISTING (2 sequences)

SEQ ID NO: 1 - protein, 37 aa, Halobacterium halobium
ACEQNPIYWARYADWLFTTPLLLLDLALLVDADEGCT

SEQ ID NO: 2 - protein, 33 aa, Halobacterium halobium
ACEDQNDPYWARYADWLFTTPLLLLDLALLVGT